(19) United States
(12) Patent Application Publication
(10) Pub. No.: US 2006/0079846 A1
(43) Pub. Date: Apr. 13, 2006

(54) Hypodermic syringes with multiple needles and methods of calming psychiatric patients using such

(76) Inventor: Alton Williams, Durham, NC (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/960,504

(22) Filed: Oct. 8, 2004

(51) Int. Cl. A61M 5/00

(52) U.S. Cl. 604/191; 604/173

(57) ABSTRACT

Hypodermic syringes with multiple needles are used to practice a method of calming psychiatric patients. In accordance with one embodiment of the invention generally used to calm violent adult patients, the syringe has a first barrel containing an antipsychotic, a second barrel containing a sedating antihistamine and a third barrel containing an antianxiety sedative. Each barrel also has a separate projecting needle and contains a piston. A common operator, preferably in the form of a plunger simultaneously pushes all of the pistons so that the patient receives three injections simultaneously. For children, a second embodiment of the syringe includes two barrels instead of three, each barrel containing a separate medication. In each embodiment the syringe is packaged in a manually openable plastic envelope with a safety cap over the needles.

### FIELD OF THE INVENTION

The present invention relates to hypodermic syringe with multiple needles and to methods of calming psychiatric patients using such. More particularly, the invention relates to administering medication to psychiatric patients during difficult situations.

### BACKGROUND OF THE INVENTION

In psychiatric in-patient units and emergency rooms, patients can become violent. On these occasions they can harm to themselves or others. In addition, they frequently resist the efforts of staff to console, and de-escalate them. When verbal reasoning fails, and violence towards others or self-destructive behavior persists, it is a psychiatric emergency. In this situation, the staff often employs use of medications which quickly sedate the violent or physically threatening patient. Many steps must occur simultaneously to sedate these patients, and this poses some risk to the staff and the patient while administering these medications.

The medications used during such an emergency, are antipsychotics (such as haloperidol), benzodiazepines (such as Lorazepam), and a sedating antihistamine (such as Diphenhydramine). Alternatively, Benztropine can be used in place of Diphenhydramine. These medications are used in combination and are administered intramuscularly (needle is inserted directly into the skin, often in the deltoid or buttocks) because the patient frequently will not agree to take these medications orally. In addition, the onset of the effects of the medications is faster when administered by intramuscularly.

The staff must do a number of things during an episode in which a patient is violent. First, several staff must hold the patient's limbs (arms and legs), to prevent the patient from attacking them or resisting administration of the medications. Second, the staff will likely place the patient in locked leather restraints (leather straps that secure the patient's arms and legs to the corners of the bed or stretcher). Third, they will then administer the medications. There is some danger in bringing a needle near a patient that is resisting the staffs efforts to restrain them. In particular, the patient can pull or push the needles into a staff member or himself.

In addition to the danger of manipulating a needle around a violent patient, there is the issue of urgency. Often times it is a nurse, nurse's aid, or mental health worker that first witnesses the violent act or aggressive act, and alerts the psychiatrist on call to make a determination as to whether there needs to be administration of medications intramuscularly. Often times the psychiatrist on call is not immediately present on the scene, and it may take time for the psychiatrist on call to establish contact with the unit. During this period the staff on the scene must physically restrain the patient, and a nurse may preemptively draw up medications to be ready should the psychiatrist on call order them. In these circumstances only the registered nurses are authorized to draw up and administer these medications. However, the nurse is often physically involved in restraining the patient, or helping the nurse's aids or mental health workers in the process, while assuming a leadership roll until the psychiatrist on call arrives. In these situations the nurse has to leave the scene, go to the medicine room, and take time to draw up the medications in needle(s), and then return to the bed where the patient is being restrained. This deprives the staff of one person for some time.

Currently a nurse must spend considerable time locating and opening packaging and drawing several medications into one or multiple syringes. The nurse then has to push the plunger on each syringe, and pay attention to the needles already injected. Often this is done while the patient is struggling. Thus current practice has substantial drawbacks.

### SUMMARY OF THE INVENTION

In view of the aforementioned considerations, a syringe useful in the treatment of a psychiatric patient comprises multiple barrels, each containing a different medication, each having a piston therein, and each having a hypodermic needle projecting therefrom. The barrels are mounted in a single housing having first and second ends, the housing enclosing the chambers with the needles projecting from the first end of the housing. An operator associated with the housing is coupled to the pistons for simultaneously pushing the pistons into the chambers toward the needles to inject the medications substantially simultaneously into the patient.

In a further aspect, some medications utilized, are an antipsychotic, a sedating antihistamine and an antianxiety sedative.

In a further aspect of the syringe, a readily removable protective cap is positioned over the needles and the syringe is in combination with a manually openable plastic package.

In a further aspect of the syringe, the antipsychotic is Haloperidol, Chlorpromazine, Ziprasidone, Risperidone, Quetiapine or Aripiprazole and the sedating medications are Lorazepam and Diphenhydramine. In addition, Benztropine and Diphenhydramine are used for prophylaxis of potential side effects of the antipsychotic. Thus, Benztropine is sometimes used instead of Diphenhydramine.

In still a further aspect of the syringe, the needles are of different internal diameters and in still another aspect of the invention, the operator associated with the housing is a plunger projecting from a second end of the housing and coupled to the pistons within the chambers to push the pistons upon being manually pressed into the housing.

The invention is also directed to a method for treating a violent and struggling patient comprising injecting the patient simultaneously with an antipsychotic, and sedating medications from one syringe through at least three needles at spaced sites on the patient.

In still a further aspect of the invention, the medications injected are Haloperidol, Chlorpromazine, Olanzapine, Lorazepam, Chlordiazepoxide, Ziprasidone, Risperidone, Quetiapine or, Aripiprazole, Diphenhydramine or Benztropine.

### BRIEF DESCRIPTION OF THE DRAWINGS

Various other features and attendant advantages of the present invention will be more fully appreciated as the same becomes better understood when considered in conjunction with the accompanying drawings, in which like reference characters designate the same or similar parts throughout the several views, and wherein:

FIG. 1 is a side view of a first embodiment of the syringe configured in accordance with the principles of the present invention;

FIG. 2 is a side view of a second embodiment of a syringe configured in accordance with the principles of the present invention, and

FIG. 3 is a side view of the syringe of FIG. 1 or 2 packaged prior to use.

### DETAILED DESCRIPTION

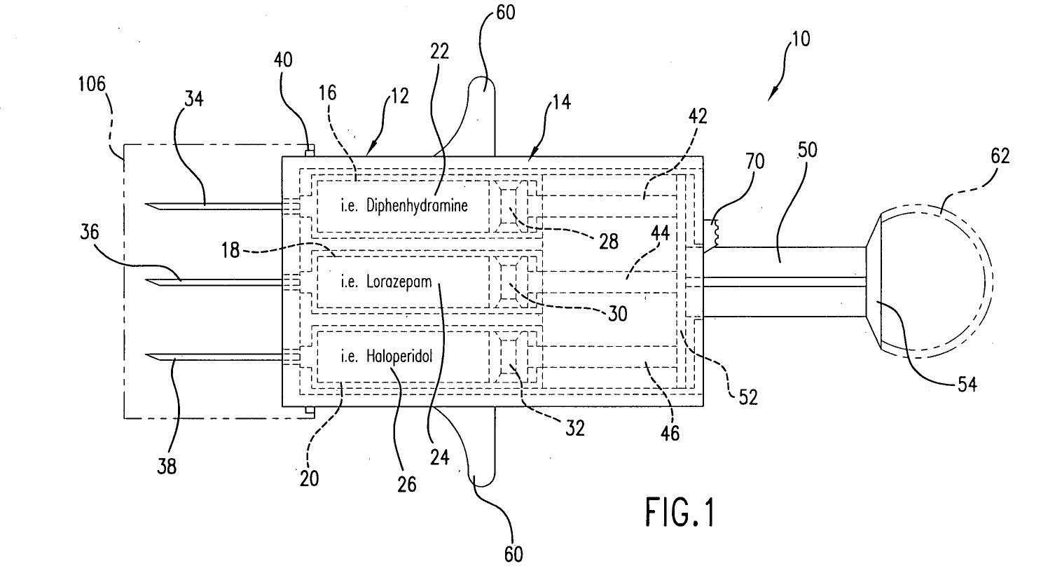

Referring now to FIG. 1 there is shown a first embodiment 10 of a syringe 12 configured in accordance with the principles of the present invention wherein the syringe 12 comprises a housing 14 containing therein three cylindrical syringe barrels 16, 18 and 20. Each cylindrical syringe barrel 16, 18 and 20 contains a different medication 22, 24 and 26 and separate pistons 28, 30 and 32, respectively for ejecting the medications from the barrels. Separate needles 34, 36 and 38 project from the barrels 16, 18 and 20, respectively out of a first end 30 of the housing 14. The barrels, pistons therein and needles projecting therefrom, are in a preferred embodiment of the syringe 10 separate hypodermic injectors which are mounted together in the single housing 14 in order to operate simultaneously. Each of the barrels 16, 18 and 22 has a front end that abuts a stop 30 within the housing so that as the pistons 28, 30 and 32 are pushed, the three medications 22, 24 and 26 are ejected through the needles 34, 36 and 38.

The pistons 28, 30 and 32 have piston rods 42, 46 and 48 projecting therefrom which are connected via a common coupling 50 to a plunger 52. The coupling 50 utilizes a surface which bears simultaneously against outer end portions of the piston rods 42, 44 and 46.

The plunger 50 has an end portion 54, which may be either a pad or a ring, while the housing of the syringe 14 has a pair of finger flanges 60 so that the syringe 12 can be operated with one hand with the physician or nurses forefinger and middle finger being in front of flanges 60 and the thumb pressing against the pad 54, which may be provided by a portion of a thumb ring 62. Preferably, the plunger 52 is held projected out of the housing 14 by a safety detent or other safety device 70 which can be quickly shifted out of engagement with the plunger 50 in order to allow the plunger to be pressed into the housing.

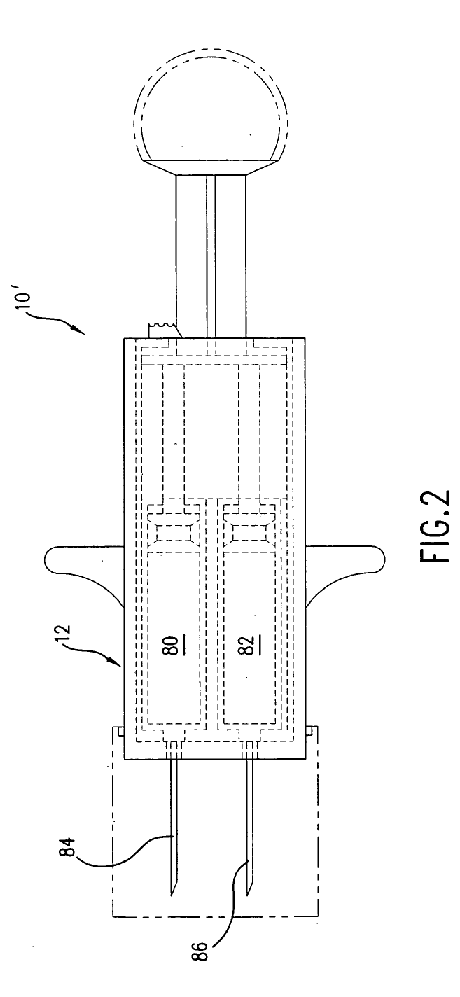

Referring now to FIG. 2 there is shown a second embodiment of the invention which includes two barrels 80 and 82 with associated needles 84 and 86 to comprise a second embodiment 10′ of the syringe 12. The second embodiment 10′ is useful in calming highly disturbed, violent children who may injure others or themselves.

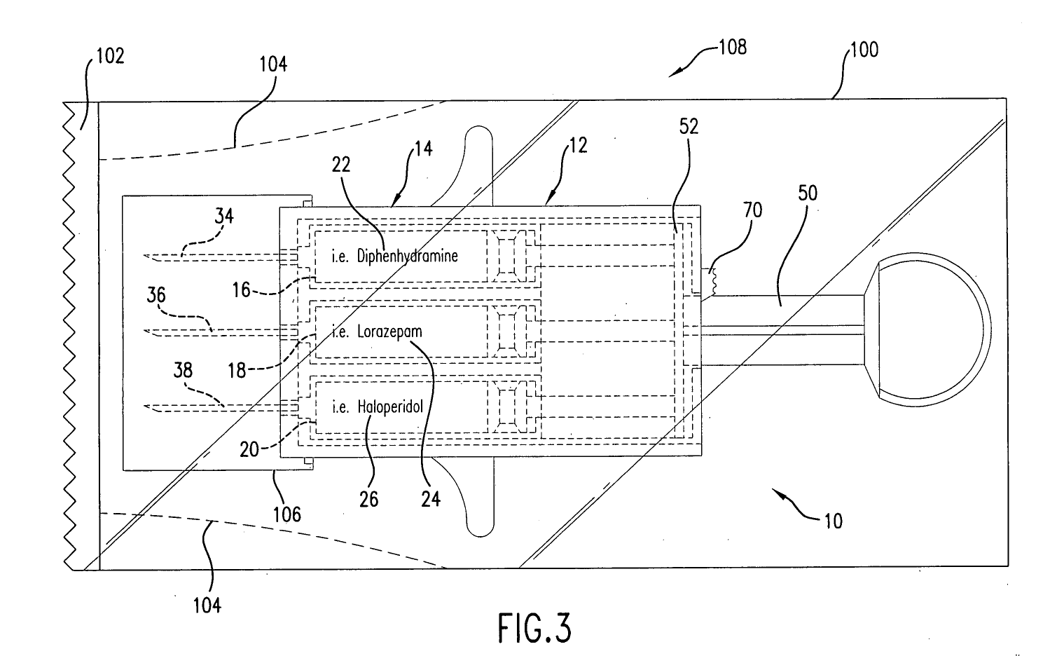

Referring now to FIG. 3 there is illustrated a preferred package for the syringes 12 of either FIG. 1 or FIG. 2 wherein the syringes are packaged in a plastic envelope 100. The plastic envelope is readily openable by tearing a creased end 102 having ridges and grooves. The tear propagates along a line 104 to expose the syringe 12. The syringe 12 is packaged with a protective cap 106 which is removed by the physician or nurse prior to use. Since the plunger 52 is extended out of the housing 14, the syringe 12 is usable immediately upon removal from the package and after removal of the cap 106. Preferably, the cap 106 snap fits with the housing 14 of the syringe so as to be readily manually removable therefrom.

The psychiatrist and nurse are thus provided a pre-packaged unit 108 with commonly used doses of medications 22, 24 and 26 frequently administered to violent or agitated psychiatric patients, which pre-packaged unit minimizes time a nurse may have to spend away from a violent situation involving a patient. By having the pre-packaged unit 108, a mental health worker or nurse's aid obtains the pre-packaged syringes 10 and brings them to the scene, thus obviating the need for a nurse to leave his or her leadership role. In addition, if the nurse is the one that retrieves the pre-packaged needles, the nurse does not have to spend time drawing-up three different medications; nor does the nurse have to manipulate three separate ampoules; nor does the nurse have to draw different medications into at least one syringe, or perhaps more than one syringe, if the medications do not fit into a single syringe. Normally the doses used for psychiatric purposes are commonly employed so the pre-packaged arrangement 108 provides a convenience to the staff during emergencies by containing a combination of medications in a single tamper proof container.

Preferably, the barrels 16, 18 and 20 are syringes already loaded with commonly used dosages of medications frequently used during psychiatric emergency situations. By having a single plastic protective cap 106 covering all three needles 34, 36 and 38, the need for a nurse to manipulate three safety caps and inadvertently stick themselves is obviated.

The syringe 12 decreases the time a nurse must spend locating and opening packaging and must spend drawing medications into one or multiple syringes. In emergency situations where it would be inconvenient for a trained nurse to leave the patient, a less trained staff member can obtain a pre-packaged syringe 12 and bring it to the nurse for administration. As stated above, the need for the nurse to handle several separate needles in close proximity to a violent patient who is often thrashing limbs in attempts to resist staff is eliminated. Moreover, additional dose errors by a nurse already in a stressful situation is minimized because the nurse does not have to draw up medications and perform the milligram to milliliter conversions required when drawing medication into syringes. In addition, if a nurse is waiting for a response from an psychiatrist on call, the nurse can be prepared immediately once given the order, as opposed to waiting for an order and then drawing up anyone of many combinations of many medications.

Commonly, violent or aggressive patients are administered 5 (mg) of Haloperidol, 1-2 mg of Lorazepam, and 50 mg of Diphenhydramine. Alternatively, 1-2 mg of Benztropine is used instead of the Diphenhydramine. In the usual situation three medications are administered to the patient. Consequently, the first embodiment 10 of the syringe 12 is used where a few medications are employed, as is the case involving adults where three medications are frequently utilized.

When violent or physically aggressive children need to be calmed, the second embodiment 10′ of the syringe having two barrels is used. As an antipsychotic, Chlorpromazine may be used, which comes in a solution that contains 25 mg of Chlorpromazine per ml. Children may receive doses of 12.5 to 50 mg. of Chlorpromazine. In addition, children may also receive Lorazepam in doses of 0.5 mg or 1 mg. Accordingly, the pediatric population requires a minimum of two different medications during episodes of violence.

Already in place are the medications in solution form. Haloperidol is available in a solution that contains 5 mg per milliliter (ml); Lorazepam is available in solutions that contain 2 mg per ml; Diphenhydramine is available in a solution that contains 25 mg or 50 mg per ml, and Benztropine is available in a solution that contains 1 mg per ml. Based on these concentrations, for the typical adult patient; a minimum of 1 ml of Haloperidol, 1 ml of Diphenhydramine, and 0.5 ml of Lorazepam (at a minimum of 2.5 ml of solution) would be needed in a syringe 12. Nurses can use a 3 cc syringe, which would hold all the medications for a minimum dosing: 5 mg of Haloperidol, 1 mg of Lorazepam, and 50 mg of Diphenhydramine. However, once the dose increases, multiple needles will likely be employed. Frequently for a greater amount of sedation 2 mg of Lorazepam is used for a large or extremely violent patient. There are larger syringes available such a s10 cc or 20 cc syringes, which could hold larger doses. However, when this is done a larger bore needle must be employed. The 3 cc needle can use a 21 or 22 gauge needle, which are small bore (small internal diameter) needles. The larger syringes can also utilize a 21 or 22 gauge needle, but the increased volume of fluid takes a longer amount of time to inject through a smaller bore 21 or 22 gauge needle. Taking a longer amount of time is not practical when struggling with a violent patient.

The dosing arrangements would come with the following combinations of medications.

| ChemStraint | | | Diphenhy- | |
|---|---|---|---|---|
| Combination | Haloperidol | Lorazepam | dramine | Benztropine |
| A | 5 mg = 1 ml | 1 mg = .5 ml | 50 mg = 1 ml | |
| B | 5 mg = 1 ml | 2 mg = 1 ml | 50 mg = 1 ml | |
| C | 5 mg = 1 ml | 1 mg = .5 ml | None | 1 mg = 1 ml |
| D | 5 mg = 1 ml | 1 mg = .5 ml | none | 2 mg = 2 ml |
| E | 5 mg = 1 ml | 2 mg = 1 ml | none | 1 mg = 1 ml |
| F | 5 mg = 1 ml | 2 mg = 1 ml | none | 2 mg = 2 ml |

It should be noted that the above combinations are not exhaustive. There are newer antipsychotic medications that are milder than Haloperidol or Chlorpromazine. Some of these newer medications include Olanzapine, Ziprasidone, Risperidone, Quetiapine, and Aripiprazole. Intramuscular preparations of Risperidone, Ziprasidone and Olanzapine are available and could be employed in the present invention.

The hypodermic syringe 12 preferably has a length of about 2.5 to 3 inches long depending on whether 1.5 or 1 inch needles 34, 36 and 38 are attached. The safety cap 106 snaps on and off to cover and uncover the three 21 or 22 gauge needles that are 1.0 or 1.5 inches in length. A 1.0 inch is preferable in children or thin adults.

From the foregoing description, one skilled in the art can easily ascertain the essential characteristics of this invention, and without departing form the spirit and scope thereof, can make various changes and modifications of the invention to adapt it to various usages and conditions.

I claim:

1. A medical syringe useful in the treatment of a patient, the syringe comprising:
a plurality of barrels each having a projecting needle and containing a separate medication,
a housing having first and second ends, the housing enclosing the barrels with the needles projecting from the first end of the housing, and
a common operator associated with the housing and coupled to the pistons for simultaneously pushing the pistons into the barrels toward the needles to inject the medications substantially simultaneously into the patient.

2. The syringe of claim 1 wherein the hypodermic needles of the syringe are of different internal diameters.

3. The syringe of claim 1 wherein each of the barrels and associated pistons and hypodermic needles are separate units assembled together in the housing and coupled to the operator.

4. The syringe of claim 3 wherein the operator is a plunger projecting from the second end of the housing and coupled with the pistons to push the pistons upon being manually pressed toward the housing.

5. The syringe of claim 4 wherein the housing is made of plastic.

6. A syringe useful in the treatment of psychiatric patients, the syringe comprising:
a first barrel containing a first medication, the first barrel having a first piston therein and a first hypodermic needle projecting therefrom;
a second barrel containing a second medication, the second barrel having a second piston therein and a second hypodermic needle extending therefrom;
a third barrel containing a third medication, the third barrel having a third piston therein and a third hypodermic needle extending therefrom;
a housing having first and second ends, the housing enclosing the barrels with the needles projecting from the first end of the housing, and
an operator associated with the housing and coupled to the pistons for simultaneously pushing the pistons into the barrels toward the needles to inject the medications substantially simultaneously into a patient.

7. The syringe of claim 6 wherein the first medication is a clinically effective dose of antipsychotic, the second medication is a clinically effective dose of a sedating antihistamine or an anticholinergic and the third medication is a clinically effective dose of an anti-anxiety sedative.

8. The syringe of claim 7 further including a readily removable protective cap over the needles.

9. The syringe of claim 8 in combination with a plastic package which is manually openable.

10. The combination of claim 9 wherein the package has a weakened portion allowing rapid opening of the package.

11. The syringe of claim 7 wherein the first medication is Haloperidol, Chlorpromazine, Olanzapine, Ziprasidone, Risperidone, Quetiapine or Aripiprazole; wherein
the second medication is Lorazepam or Chlordiazepoxide,
and the third medication is Diphenhydramine or Benztropine.

12. The syringe of claim 13 wherein the hypodermic needles of the syringe are of different internal diameters.

13. The syringe of claim 11 wherein each of the barrels and associated pistons and hypodermic needles are separate units assembled together in the housing and coupled to the operator.

14. The syringe of claim 13 wherein the operator is a plunger projecting from the second end of the housing and coupled with the pistons to push the pistons upon being manually pressed toward the housing.

15. The syringe of claim 14 wherein the housing is made of plastic.

16. A method of treating a violent and struggling psychiatric patient, comprising:
injecting the patient simultaneously with an anti-psychotic, sedating antihistamine and an antianxiety sedative from one syringe through at least three needles at spaced sites on the patient.

17. The method of claim 16 wherein the first medication is Haloperidol, chlorpromazine, Olanzapine, Ziprasidone, Risperidone, Quetiapine or Aripiprazole; wherein the second medication is Lorazepam, and wherein the third medication is Diphenhydramine or Benztropine.

18. The method of claim 16 wherein the rate of injection is varied by varying the internal diameter of the needles.

19. A syringe useful in the treatment of a child psychiatric patient, the syringe comprising:
a first barrel containing a first medication, the first barrel having a first piston therein and a first hypodermic needle projecting therefrom;
a second barrel containing a second medication, the second barrel having a second piston therein and a second hypodermic needle extending therefrom;
a housing having first and second ends, the housing enclosing the barrels with the needles projecting from the first end of the housing, and
an operator associated with the housing and coupled to the pistons for simultaneously pushing the pistons into the barrels toward the needles to inject the medications substantially simultaneously into the child psychiatric patient.

20. The syringe of claim 19 wherein the first medication is a clinically effective dose of an antipsychotic and the second medication is a clinically sedating antihistamine.

21. The syringe of claim 20 further including a readily removable protective cap over the needles.

22. The syringe of claim 21 in combination with a plastic package which is manually openable.

23. The combination of claim 22 wherein the package has a weakened portion allowing rapid opening of the package.

24. The syringe of claim 20 wherein the first medication is a clinically effective dose of chlorpromazine and wherein the second medication is a clinically effective dose of Lorazepam.

25. A method of treating a child psychiatric patient, comprising:
injecting the patient simultaneously with an anti-psychotic, and a sedating antihistamine through at least two needles at spaced sites on the patient.

26. The method of claim 25 wherein the first medication chlorpromazine and wherein the second medication is Lorazepam.

27. The method of claim 25 wherein the rate of injection is varied by varying the internal diameter of the needles.
